(19) United States
(12) Patent Application Publication
(10) Pub. No.: US 2013/0226096 A1
(43) Pub. Date: Aug. 29, 2013

(54) CARTRIDGE FOR DRUG DELIVERY DEVICES

(71) Applicants: MIchael Jugl, Frankfurt am Main (DE); Turhan Ergel, Frankfurt am Main (DE)

(72) Inventors: MIchael Jugl, Frankfurt am Main (DE); Turhan Ergel, Frankfurt am Main (DE)

(73) Assignee: SANOFI-AVENTIS DEUTSCHLAND GMBH, Frankfurt am Main (DE)

(21) Appl. No.: 13/883,395

(22) PCT Filed: Nov. 14, 2011
(86) PCT No.: PCT/EP2011/069997

§ 371 Date: May 3, 2013

(30) Foreign Application Priority Data
Nov. 16, 2010 (EP) 10191278.0

(51) Int. Cl. A61M 5/32

(52) U.S. Cl. CPC A61M 5/3286
USPC 604/201

(57) ABSTRACT

The present invention relates to a cartridge for a drug delivery device comprising of a container at least partially filled with an injectable medicament, at least one sealing member disposed at a distal end of the container, the sealing member being pierceable by a piercing element for expelling a dose of the medicament, and at least one fixing cap of circular or cylindrical geometry comprising a centric through opening at a distal end face giving access to the sealing member disposed underneath, and comprising at least in sections a structurally strengthened surface structure to keep the sealing member in position and/or to counteract a pressure-induced mechanical deformation of the sealing member, and wherein the distal end face of the fixing cap comprises at least one radially extending corrugation or bead.

### CROSS REFERENCE TO RELATED APPLICATIONS

The present application is a U.S. National Phase Application pursuant to 35 U.S.C. §371 of International Application No. PCT/EP2011/069997 filed Nov. 14, 2011, which claims priority to European Patent Application No. 10191278.0 filed Nov. 16, 2010. The entire disclosure contents of these applications are herewith incorporated by reference into the present application.

### FIELD OF DISCLOSURE

The present invention relates to a cartridge for drug delivery devices and in particular two cartridges to be used with injection devices, such like pen-type injectors. The cartridge is typically related to such injectors, where a user may set and dispense the dose by himself.

### BACKGROUND

User operated drug delivery devices are typically applicable in circumstances, where persons without formal medical training, in particular patients, need to administer an accurate and predefined dose of a medicament, such as heparin or insulin. Such devices are typically applied, where the medicament is to be administered on a regular or irregular basis of a short or long-term period.

Generally, such devices should be robust in construction, jet easy to use in terms of handling and in understanding by the user of its operation and the delivery of the required dose. Furthermore, the dose setting must be easy and unambiguous. When the device is to be disposable rather than reusable, it should be inexpensive to manufacture and easy to dispose. To meet these requirements, the overall number of parts required to assemble the device should be kept on a minimum.

The injectable medicament to be dispensed by way of such drug delivery devices is typically provided in a disposable or replaceable cartridge, such as a vial, ampoule or carpule comprising a barrel, typically of cylindrical geometry, and a piston slidably disposed therein for sealing the cartridge. While a proximal end of the cartridge is provided with the piston to be operably engaged with a piston rod of the drug delivery device's drive mechanism, opposite, at its distal end section, the cartridge is typically sealed by a sealing member which is to be pierced by a piercing element, like an injection needle or cannula in order to establish a fluid transferring coupling allowing to dispense the medicament.

This way, by applying thrust to the cartridge's piston in distal direction, a pre-defined dose of the liquid medicament can be dispensed and expelled from the cartridge in a well-defined way.

The distal sealing member, commonly denoted as septum is typically designed as a rubber stopper providing an air-tight seal to be pierced and penetrated by the piercing element.

In order to provide an air-tight but pierceable seal, the septum is made of a flexible and mechanically deformable material. In the course of dose dispensing, a respective fluid pressure in the inner volume of the cartridge is built-up, which, due to the elastic properties of the septum may lead to a respective expansion or elastic deformation of the septum. Consequently, the septum may expand into or even through an aperture provided in a fixing cap which in turn is intended to keep the septum in its sealing position at the distal end of the cartridge.

By such pressure induced expansion, prolongation or general deformation, the distally located sealing member inevitably stores elastic energy during a dose dispensing procedure. Typically, when after termination of the dose dispensing procedure the fluid pressure inside the cartridge returns to an initial and lower value, the sealing member relaxes into its initial shape. This relaxation process is typically accompanied by a retraction of the previously expanded section of the sealing member back into the cartridge. However, such retracting motion may in turn lead to another built-up of a non-negligible fluid pressure inside the cartridge and as a consequence, a certain amount of the medicament may be supplementally expelled from the cartridge.

It is therefore mainly due to the elastic properties of the sealing member that within a time interval after termination of the dose dispensing procedure a droplet formation can be observed at the distal tip of the piercing element.

It is therefore an object of the present invention, to provide an improved cartridge for a drug delivery device that counteracts generation of droplets after termination of a dose dispensing procedure. Furthermore, the invention aims to provide an effective means adapted to prevent or at least to counteract septum deformation during dose dispensing. With these demands, it is further intended, to provide an inexpensive as well as a stable and robust design for such medicament-containing cartridges.

### SUMMARY

According to a first aspect, a cartridge for a drug delivery device is provided that comprises a container, typically in form of a cylindrical barrel, being at least partially filled with an injectable medicament. The container is sealed by at least one sealing member disposed at a distal end of the container. The sealing member is pierceable by a piercing element for expelling a dose of the medicament and inherently comprises a specific elasticity in order to provide a substantially air-tight seal, in particular when the piercing element and the container are de-coupled, hence, when the sealing member is pulled out of the sealing member.

The cartridge further comprises at least one fixing cap having at least in sections a structurally strengthened surface structure in order to keep the sealing member in a pre-defined position with respect to the container and/or to counteract a pressure-induced mechanical deformation of the sealing member. By structurally strengthening the fixing cap and/or at least its surface structure, the stiffened fixing cap may serve to stabilize the sealing member disposed underneath. In particular, by enhancing the rigidity and stiffness of the fixing cap, axial expansion and elastic deformation of the sealing member can be advantageously reduced without modifying the geometric and/or elastic properties of the sealing member itself.

By structurally strengthening the fixing cap, the whole entity comprised of sealing member and fixing cap can be structurally supported and enhanced. This way, the sealing member's liability to pressure induced deformation can be reduced and as a consequence droplet generation can be constricted.

The fixing cap is of circular or cylindrical geometry and further comprises a centric through opening or an aperture at a distal end face that gives access to the sealing member disposed underneath. By way of its distal end face, the fixing cap is adapted to fix the sealing member in axial direction with respect to the cylindrical geometry of the cartridge. The centric through opening of circular or oval, even rectangular or quadratic geometry gives direct access to the sealing member disposed underneath in order to pierce the same by means of a piercing element.

In other words, the fixing cap embraces a distal outlet end of the cartridge and comprises a radially inwardly extending flange portion that serves to keep the at least one sealing member in position.

In particular, the distal end face of the fixing cap comprises radially extending corrugations or beads. These radially extending stiffening surface structures may extend in radial direction, for instance across the entire radial width of the distal end face and/or its radially inwardly directed flange portion. Preferably, the radially extending corrugations or beads are arranged in a regular evenly distributed manner across the distal end face of the fixing cap. If for instance three radially extending corrugations are provided, they will preferably extend at an angle of 120°. With four corrugations, the circumferential angle at which neighbouring corrugation extend may reduce to 90°.

According to another aspect, the structurally strengthened fixing cap embraces a distal neck portion of the container. The container, typically made of glass or a comparable transparent material comprises a stepped-down neck portion at its distal end featuring a radially extending head forming an undercutting or indentation adapted to positively engage with the fixing cap for holding and fixing the same with the container.

In another preferred embodiment, the fixing cap comprises a ferrule of sheet metal. Typically, the fixing cap comprises a ferrule made of aluminum and is therefore adapted to be fixed to the distal head or outlet section of the container by way of a crimping procedure or by means of similar mechanical deformations.

Consequently, and according to another preferred embodiment, the distal end face of the fixing cap at least in sections comprises at least one corrugated or beaded structure or surface portion to provide a structural strengthening of the distal end face. Hence, by introducing corrugations, beads, ribs or the like reinforcing fins or seams, the rigidity and stiffness of the fixing cap, in particular of its distal end face can be enhanced so as to limit pressure induced mechanical deformation of the sealing member.

By providing corrugated or beaded surface portions on the fixing cap, the rigidity and stiffness of the cap can be modified in sections according to predefined requirements.

Generally, by way of the corrugations or beads in the distal end face of the fixing cap, enhanced stiffness and rigidity can be attained without the necessity of making use of different or differently shaped materials for the fixing cap. Moreover, the general geometry of the cap can remain unaltered and the amount of material required to produce a fixing cap can be kept substantially on the same level compared to conventional fixing cap designs.

Alternatively or additionally, the distal end face of the fixing cap comprises at least one circumferentially extending corrugation or bead. This structurally strengthening structure may be concentrically disposed with respect to the position and/or shape of the centric through opening of the distal end face. The circumferentially extending corrugations or beads may comprise an annular and closed structure. If two or several corrugations or beads are provided, they mutually distinguish by different radii.

In still another aspect, the distal end face of the fixing cap comprises mutually intersecting stiffening corrugations or beads that form a structurally stiffening pattern on the distal end face of the fixing cap. Depending on the shape and position of respective corrugations or beads, crosswire or crosshair structures as well as hexagonal or rhombus-like stiffening structures are conceivable.

Furthermore and according to another independent aspect, the stiffening corrugations or beads may axially protrude in distal direction from the distal end face of the fixing cap. Alternatively or additionally, all or at least some of said corrugations may also be embossed or engraved in the distal end face and may therefore form a kind of indentation in the outer surface of the fixing cap.

Moreover and according to a further embodiment, the at least one corrugation or bead, preferably all corrugations and/or beads extend outside the centric through opening of the fixing cap. Hence, the at least one corrugation or bead, preferably all structurally strengthened surface portions of the fixing cap are located outside the centric through opening of the fixing cap. The distal end face of the fixing cap comprises a radially inwardly extending flange portion which is structurally strengthened and which comprises at least one corrugation or bead.

Radially inwardly said flange portion delimits and confines the through opening provided in the distal end face of the fixing cap. By structurally strengthening the radially inwardly extending distal flange portion of the fixing cap in a selective and purposeful way, the distal end face of the fixing cap becomes less prone to mechanical deformation and may therefore counteract formation of a bulged portion of the septum located underneath the fixing cap.

Preferably, the through opening or the aperture of the fixing cap is free of any structurally strengthening surface structures like corrugations or beads. Any such structurally enhancing structures entirely extend in the ring-like flange portion of the cap extending radially inwardly from a radial outer edge of the cap towards the radial inner edge adjacent to the through opening.

According to another but independent aspect, the invention also refers to a drug delivery device for dispensing of a dose of an injectable medicament. The drug delivery device comprises a housing and a cartridge as described above which is disposed in said housing and which is sealed in proximal direction by means of a piston slidably disposed in the cartridge. The drug delivery device further comprises a drive mechanism having a piston rod operably engaged with the piston of the cartridge in order to expel a dose of the medicament from the cartridge during a dose dispensing action.

The term “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, a antibody, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(02)25, Asp28] Exendin-4(1-39),
des Pro36 [Met(O)14 Trp(02)25, IsoAsp28] Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39),
wherein the group -Lys6-NH2 may be bound to the C-terminus of the Exendin-4 derivative;
   or an Exendin-4 derivative of the sequence
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(O2)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(O2)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;
or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the pertinent art that various modifications and variations can be made to the present invention without departing from the spirit and scope of the invention. Further, it is to be noted, that any reference signs used in the appended claims are not to be construed as limiting the scope of the present invention.

### BRIEF DESCRIPTION OF THE DRAWINGS

In the following, preferred embodiments of the invention will be explained in greater detail by making use to the drawings in which:

FIG. 1 shows a schematic side view of a cartridge,

FIG. 2 illustrates an enlarged view of the distal outlet end of the cartridge and

FIG. 3 schematically illustrates the distal end of the cartridge in a perspective illustration.

### DETAILED DESCRIPTION

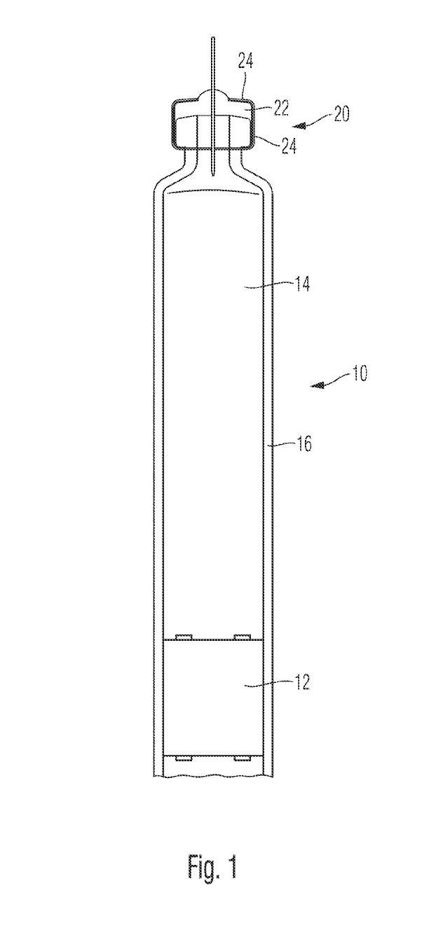

The cartridge 10 as illustrated in FIG. 1 comprises a container 16 of cylindrical geometry, which is at least partially filled with a liquid medicament 14. The proximal end of the barrel-like container 16 is sealed by a piston 12 slidably disposed in the container 16 in axial direction, that is upwards or downwards in FIG. 1, along the container's 16 longitudinal axis.

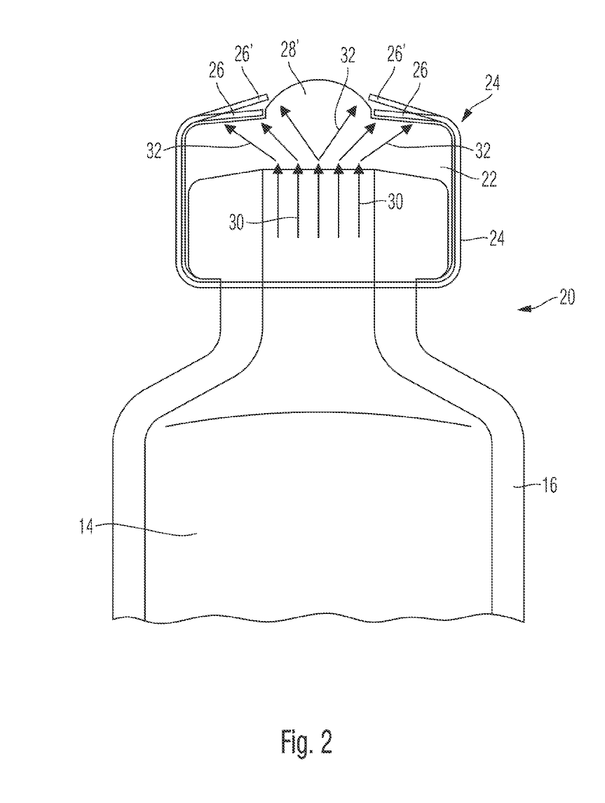

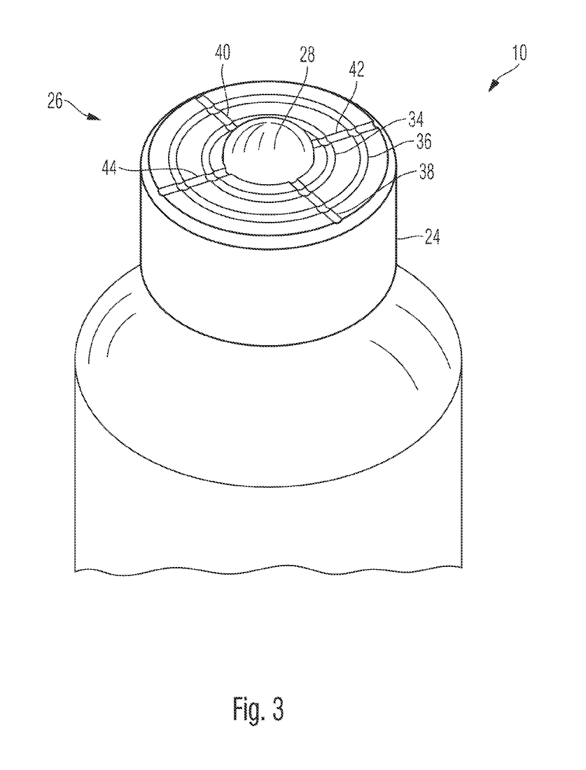

The upper section of the cartridge 10 as illustrated in FIG. 1 is schematically shown in FIGS. 2 and 3 in an enlarged view. The container 16 at its upper, distal end section comprises a neck portion 20 and a radially extending head section being entirely embraced by a fixing cap 24, typically made of deformable sheet metal, such like aluminum. The fixing cap 24 is typically crimped over the outlet end of the glass container 16. At its upper or distal end face 26, the fixing cap 24 comprises a through opening providing access to a centric portion 28 of the sealing member 22 being disposed between an inside facing wall section of the fixing cap 24 and the distal end face of the head of the container 16.

In the sketch of FIG. 2, the upward pointing arrows 30 represent a fluid pressure or inner pressure which builds up during a dose dispensing action, i.e. when the piston 12 is forced in a distal direction, which means upward in FIG. 1.

Since the sealing member or the septum 22 has to be made of an elastic material in order to provide an air-tight but multiple pierceable seal, said inner pressure leads to a respective built-up of mechanical stress 32 inside or across the sealing member 22. As a consequence and as illustrated in FIG. 2, the inner pressure-built up inside the container 16 leads to the formation of a bulged portion 28′ of the sealing member 22 at least partially reaching through the central aperture of the fixing cap 24 and partially protruding from its distal end face 26. As a consequence of this bulge formation 28′ also the radially inwardly extending flange portion 26′ of the fixing cap 24 can be at least slightly bended in distal, hence upward direction as illustrated in FIG. 2.

By providing the fixing cap 24 and at least its distal end face 26 with radially and/or circumferentially extending corrugations or beads 34, 36, 38, 40, 42, 44, as illustrated in FIG. 3, the distal end face 26 and the sealing cap 24 can be structurally strengthened and stiffened and as a consequence, the bending of the flange portion 26′ as illustrated in FIG. 2 can be at least reduced or even entirely prevented.

By providing a structurally and mechanically strengthened distal end face 26 of the fixing cap 24, bulge formation 28′ as illustrated in FIG. 2 can be substantially reduced. This way, overall deformation of the sealing member 22 during a dose dispensing action can be attenuated with the effect, that a reduced amount of elastic energy is stored in the sealing member 22. Consequently, reduction of elastic deformation of the sealing member 22 reduces the tendency and susceptibility of the cartridge 10 with respect to post-dispending droplet generation.

It is further to be mentioned here, that the illustrated pattern of four radially extending corrugations or beads 38, 40, 42, 44 as well as the two illustrated circumferential annular corrugations or beads 34, 36 is only exemplary. Depending on the overall geometry of the sealing member and the head of the cartridge 10 as well as depending on the choice of materials used for the fixing cap 24 and the sealing member 22, position, shape and geometry as well as number and intersections of the illustrated corrugations or beads 34, 36, 38, 40, 42, 44 may vary accordingly. Further, it is to be noted, that the corrugations or beads may either be embossed as a kind of indentation into the end face 26 or may protrude there from.

In particular, the radially extending corrugations 38, 40, 42, 44 may provide a stiffening and reinforcement of the distal end face 26 of the fixing cap 24. Moreover, the concentrically arranged and circumferentially extending corrugations 34, 36 may counteract a distally directed widening of the distal end face 26 as depicted with reference numeral 26′ in FIG. 2. Such distally directed bending of the distal end face 26 would also imply a circumferential extension of the circumferential corrugations 34, 36. By way of substantially impeding a further elongation of such circumferential corrugations 34, 36, e.g. by making use of an appropriately designed corrugation or bead 34,36, a distally directed bending of the distal end face 26 can be substantially prevented, at least counteracted.

1-9. (canceled)

10. A cartridge for a drug delivery device comprising:
a container at least partially filled with an injectable medicament,
at least one sealing member disposed at a distal end of the container, the sealing member being pierceable by a piercing element for expelling a dose of the medicament, and
at least one fixing cap of circular or cylindrical geometry comprising a centric through opening at a distal end face giving access to the sealing member disposed underneath, and comprising at least in sections a structurally strengthened surface structure to keep the sealing member in position and/or to counteract a pressure-induced mechanical deformation of the sealing member, and
wherein the distal end face of the fixing cap comprises at least one radially extending corrugation or bead.

11. The cartridge according to claim 10, wherein the fixing cap embraces a distal neck portion of the container.

12. The cartridge according to claim 10, wherein the fixing cap comprises a ferrule of sheet metal.

13. The cartridge according to claim 10, wherein the distal end face of the fixing cap at least in sections comprises a corrugated or beaded structure to provide a structural strengthening of the distal end face.

14. The cartridge according to claim 10, wherein the distal end face of the fixing cap comprises at least one circumferentially extending corrugation or bead.

15. The cartridge according to claim 10, wherein the distal end face of the fixing cap comprises mutually intersecting stiffening corrugations or beads forming a structurally stiffening pattern.

16. The cartridge according to claim 10, wherein the stiffening corrugations axially protrude in distal direction and/or are embossed in the distal end face.

17. The cartridge according to claim 10, wherein the at least one corrugation or bead extends outside the centric through opening of the fixing cap.

18. A drug delivery device for dispensing a dose of an injectable medicament, comprising:
a housing,
a cartridge according to any one of the preceding claims disposed in said housing and being sealed in proximal direction by means of a piston slidably disposed therein, and
a drive mechanism comprising a piston rod being operably engaged with the piston to expel a dose of the medicament from the cartridge.
